(19) United States
(12) Patent Application Publication
(10) Pub. No.: US 2024/0260894 A1
(43) Pub. Date: Aug. 8, 2024

(54) ALLERGEN REACTION BIOFEEDBACK SYSTEMS AND METHODS

(71) Applicant: Willow Laboratories, Inc., Irvine, CA (US)

(72) Inventor: Gregory A. Olsen, Lake Forest, CA (US)

(21) Appl. No.: 18/431,389

(22) Filed: Feb. 2, 2024

Related U.S. Application Data
(60) Provisional application No. 63/483,076, filed on Feb. 3, 2023.

(51) Int. Cl. A61B 5/00; A61B 5/0205; A61B 5/145; A61B 5/1455; A61B 5/291; A61B 5/296; A61M 5/142; G16H 10/60; G16H 40/63; G16H 50/30

(52) U.S. Cl. CPC A61B 5/4836; A61B 5/0205; A61B 5/681; A61B 5/7282; A61B 5/742; A61B 5/746; A61B 5/747; A61M 5/14244; G16H 10/60; G16H 40/63; G16H 50/30; A61B 5/14532; A61B 5/14551; A61B 5/291; A61B 5/296; A61B 2560/045; A61B 2562/0204; A61B 2562/0219

(57) ABSTRACT

A system which provides example systems and methods for tracking physiological measurements of a person, computing a ranking, and/or calling to action to teach the person about their food sensitivity and the severity of the sensitivity, as well as warn the person, offer alternate substitutions, or suggest ingredients to counter their reaction. One or more sensors may be configured to measure indications of allergic reactions in a patient's body. A hardware processor may be configured to receive measurements from the one or more sensors, determine a response action, and carry out the response action. The response action can include triggering an auto-injection of epinephrine.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims the benefit of priority to U.S. Provisional Patent Application No. 63/483,076, filed Feb. 3, 2023, which is incorporated by reference herein in its entirety.

### FIELD

The general field of this disclosure is allergen monitoring and management.

### BACKGROUND

An allergic reaction occurs when a person's immune system identifies and reacts to a normally non-threatening substance as a threat to the human body. Food allergy in particular affects an estimated 8% of children under age 5 and up to 4% of adults. While there's no cure, some children outgrow their food allergies as they get older. Most allergic reactions occur within hours to two weeks especially if a person has been sensitized to that allergen previously. However, some allergic reactions may take up to six weeks to develop.

Because allergic reactions can vary in severity from mild intolerance to life threatening, it is important to identify a potential allergen exposure in order to administer treatment if necessary. Outside of severe cases where allergic reaction is often readily and immediately apparent after an allergen exposure, identification of an allergic reaction due to an allergen exposure is typically limited to patient or clinical observation through controlled exposure (or elimination of exposure) to common allergens or through blood tests to check for antibodies. However, these methods can be invasive or require long periods of time and observation in order to identify an allergic reaction or the identification of an allergen comes after a severe response. Thus, it would be advantageous to be able to identify an allergic reaction concurrently or even prior to an allergen exposure. In particular, in the case of severe and sudden allergic reaction, a way to identify potentially serious allergen exposure prior to or in concurrence with an allergic reaction can help with administration of life saving injection of medication. Additionally, because allergic reactions can increase in severity with repeated exposure, early detection of allergic sensitivity and reactions prior to when most patients would seek medical diagnosis of an allergic reaction can be helpful in avoiding more severe allergic reactions in the body.

### SUMMARY

Various embodiments of systems, methods and devices within the scope of the appended claims each have several aspects, no single one of which is solely responsible for the desirable attributes described herein. Without limiting the scope of the appended claims, some prominent features are described herein.

In some embodiments, the systems, methods and devices herein can include a system for determining an allergic sensitivity of a patient, the system comprising at least one sensor configured to measure one or more physiological parameters of a patient, an input configured to receive at least one signal from the at least one sensor, and at least partial user history of the patient, and one or more hardware processors configured to process the at least one signal from the at least one sensor and the at least partial user history of the patient, establish a baseline physiological state of the patient or a response to at least one first potential allergen, identify a change in a physiological state of the patient or a response to at least one second potential allergen, determine an allergic reaction of the patient based on the change in the physiological state of the patient or a difference between the response to the at least one first potential allergen and the response to the at least one second potential allergen, determine a response action to the allergic reaction of the patient, and implement the response action.

In some embodiments, the at least one first potential allergen is at least one food item. In some embodiments, the at least one second potential allergen is at least one food item. In some embodiments, the at least one first potential allergen is at least one medication. In some embodiments, the at least one second potential allergen is at least one medication. In some embodiments, the at least one first potential allergen is at least one animal fur, skin, saliva or urine. In some embodiments, the at least one second potential allergen is at least one animal fur, skin, saliva or urine. In some embodiments, the at least one first potential allergen is at least one pollen. In some embodiments, the at least one second potential allergen is at least one pollen. In some embodiments, the at least one first potential allergen is at least one dust. In some embodiments, the at least one second potential allergen is at least one dust. In some embodiments, the response action comprises causing a medication injection device to inject the patient with medication. In some embodiments, the medication injection device is a wearable pump configured to inject the patient with epinephrine. In some embodiments, the medication injection device is a wearable pump configured to inject the patient with an antihistamine. In some embodiments, the medication injection device is a wearable pump configured to inject the patient with hydrocortisone. In some embodiments, the response action comprises instructing the patient to take a medication. In some embodiments, the medication comprises epinephrine. In some embodiments, the medication comprises an antihistamine. In some embodiments, the medication comprises hydrocortisone. In some embodiments, the response action comprises instructing the patient to drink water. In some embodiments, the at least one sensor comprises at least one wearable sensor. In some embodiments, the at least one wearable sensor comprises a smartwatch. In some embodiments, the at least partial user history of the patient comprises medical records. In some embodiments, the at least partial user history of the patient comprises one or more prior allergic reactions. In some embodiments, the at least partial user history of the patient comprises previously known allergens of the patient. In some embodiments, the at least partial user history of the patient comprises a history of asthma, cardiovascular disease, or chronic lung disease. In some embodiments, the baseline physiological state of the patient comprises a nonreactive state. In some embodiments, the baseline physiological state of the patient comprises a state in which the patient has not contacted potential allergens. In some embodiments, the one or more hardware processors is configured to determine the patient has an allergic reaction when the difference between the response to the at least one first potential allergen and the response to the at least one second potential allergen is greater than an allergy threshold. In some embodiments, the one or more hardware processors is configured to determine the patient has an allergic reaction when the change in the physiological state of the patient is greater than an allergy threshold. In some embodiments, the one or more hardware processors is configured to characterize a severity of the allergic reaction based on whether the difference between the response to the at least one first potential allergen and the response to the at least one second potential allergen is greater than one or more of a plurality of severity thresholds. In some embodiments, the one or more hardware processors is configured to characterize a severity of the allergic reaction based on whether the change in the physiological state of the patient is greater than one or more of a plurality of severity thresholds. In some embodiments, the response action comprises alerting the patient of the allergic reaction. In some embodiments, alerting the patient of the allergic reaction comprises notifying the patient on a display of a user device. In some embodiments, alerting the patient of the allergic reaction comprises notifying the patient on a display of a smartwatch. In some embodiments, the response action comprises alerting a contact of the patient of the allergic reaction. In some embodiments, the response action comprises alerting an emergency service of the allergic reaction. In some embodiments, the at least one sensor comprises imaging sensors, photoplethysmography sensors, acoustic sensors, electromyogram sensors, electroencephalogram sensors, or motion sensors. In some embodiments, the one or more physiological parameters of the patient comprises respiration rate. In some embodiments, the one or more physiological parameters of the patient comprises throat diameter. In some embodiments, the one or more physiological parameters of the patient comprises bodily sounds. In some embodiments, the one or more physiological parameters of the patient comprises non-invasive blood pressure. In some embodiments, the one or more physiological parameters of the patient comprises pulse rate. In some embodiments, the one or more physiological parameters of the patient comprises hydration. In some embodiments, the one or more physiological parameters of the patient comprises galvanic skin response. In some embodiments, the one or more physiological parameters of the patient comprises pulse transit time. In some embodiments, the one or more physiological parameters of the patient comprises parameters measured by an electrocardiogram. In some embodiments, the one or more physiological parameters of the patient comprises blood pressure. In some embodiments, the one or more physiological parameters of the patient comprises hemoglobin. In some embodiments, the one or more physiological parameters of the patient comprises oxygen content fractional oxygen saturation dis-hemoglobins. In some embodiments, wherein the one or more physiological parameters of the patient comprises metabolic rate. In some embodiments, the one or more physiological parameters of the patient comprises visual observation related behaviors and parameters. In some embodiments, the one or more physiological parameters of the patient comprises temperature of skin. In some embodiments, the one or more physiological parameters of the patient comprises internal temperature. In some embodiments, the one or more physiological parameters of the patient comprises appearance of hives or rash. In some embodiments, the one or more physiological parameters of the patient comprises parameters related to symptoms of allergic reactions.

In some embodiments, the systems, methods and devices herein can include a method of determining an allergic sensitivity of a patient, the method comprising receiving at least one signal from at least one sensor configured to measure one or more physiological parameters of the patient, receiving at least partial user history of the patient, processing the at least one signal from the at least one sensor and the at least partial user history of the patient, establishing a baseline physiological state of the patient or a response to at least one first potential allergen, identifying a change in a physiological state of the patient or a response to at least one second potential allergen, determining an allergic reaction of the patient based on the change in the physiological state of the patient or a difference between the response to the at least one first potential allergen and the response to the at least one second potential allergen, determining a response action to the allergic reaction of the patient, and implementing the response action.

In some embodiments, the systems, methods and devices herein can include a method for determining an allergic reaction of a patient, the method comprising receiving at least one first signal from at least one sensor configured to measure physiological parameters of the patient, receiving at least partial user history of the patient, displaying a graphical user interface (GUI) on a screen of a user device coupled to at least one sensor, the GUI containing at least one physiological parameter at a baseline physiological state of the patient or in response to at least one first potential allergen, receiving at least one second signal from at least one sensor configured to measure physiological parameters of the patient, displaying a GUI on a screen of a user device coupled to at least one sensor, the GUI containing an indication of presence of an allergic reaction of the patient, at least one physiological parameter at a baseline physiological state of the patient or in response to at least one first potential allergen, at least one physiological parameter at a changed physiological state of the patient or in response to at least one second potential allergen, and an indication of severity of the allergic reaction of the patient, wherein the user device comprises a computer processor and an electronic storage medium.

In some embodiments, the systems, methods and devices herein can include a medication injection device, the medication injection device comprising a wearable pump housing configured to receive a medication container, the medication container configured to be inserted into the wearable pump housing, the medication container having a dispensing end through which medication is dispensed, wherein the dispensing end of the medication container is configured to align with a frontal end of the wearable pump housing when the medication container is inserted into the wearable pump housing, a piston rod at a distal end of the wearable pump housing, a battery inside the wearable pump housing, the battery configured to supply electrical power to a piston rod pushing system, a hardware processor in communication with the piston rod pushing system, the hardware processor configured to determine when a patient requires medication for an allergic reaction, and the piston rod pushing system connected to the piston rod, the piston rod pushing system configured to communicate with the hardware processor to receive instructions, the piston rod pushing system further configured to push the piston rod toward the frontal end of the wearable pump housing when the hardware processor determines the patient requires medication for the allergic reaction, causing the medication to be dispensed. In some embodiments, the medication comprises epinephrine. In some embodiments, the medication comprises an antihistamine. In some embodiments, the medication comprises hydrocortisone. In some embodiments, the hardware processor determines the allergic reaction based on input from at least one sensor.

### BRIEF DESCRIPTION OF THE DRAWINGS

FIG. 1 illustrates an example environment in which an allergen detection system may operate;

FIG. 2 illustrates an example process for identifying an allergen based on physiological data;

FIG. 3 illustrates an example process of the decision logic processing signals from one or more sensors to determine an output;

FIG. 4 illustrates an example process of using sensors to determine the presence of an allergic reaction and injecting a patient with medication;

FIG. 5 illustrates an example process of using sensors to determine the presence of an allergic reaction and alerting a patient or calling for help; and

FIG. 6 illustrates an example process of using neural networks or machine learning to identify allergic reactions.

### DETAILED DESCRIPTION

A system to track physiological measurements of a person, compute a ranking, and/or call to action to teach the person about their food sensitivity and the severity of the sensitivity, as well as warn the person, offer alternate substitutions, or suggest ingredients to counter their reaction. In preparation for severe cases of reaction, an individual could have a medication management device, such as a wearable pump, with an automatic trigger to deliver a life saving injection of a medication such as epinephrine.

Current technologies can require a person undergoing an allergic reaction to determine whether they require medication or help. Current technologies can also require that the medication be administered by the person undergoing the allergic reaction or another person. Advantageously, the systems and methods described herein can allow for an automatic response to symptoms of an allergic reaction. The systems and devices described herein can provide a more timely and reliable notice of an allergic reaction. The systems and devices described herein can provide near instantaneous treatment of an allergic reaction.

In some implementations, the system may include a food logging application that allows for manual and/or automatic food tracking (such as from a planned food tray, camera recording, QR code, or smart tray) which can be paired with a smart device (such as, but not limited to, a smart watch, shirt or the like). These smart sensors (which can optionally include, but are not limited to light based reflective/transmissive, acoustic, radio, impedance or the like) can collect information such as respiration rate, pulse rate, blood pressure, methemoglobin, EKG, heart rate variability, pulse rate variability, temperature, hydration, galvanic skin response, or others. After each food consumption event, several biomarkers can be measured continuously (or sequentially) to track physiological response. Once the normal non-reactive response and patterns are known at rest, they can be compared to delta changes after ingestion or exposure to food as quantified measurements that can then be weighted and ranked to allow a software interface (Smart phone, watch, VR/AR or the like) to present an actionable insight for the wearer or their caregiver or even trigger emergency response with a direct cellular connection.

### Example Allergen Identification Environment

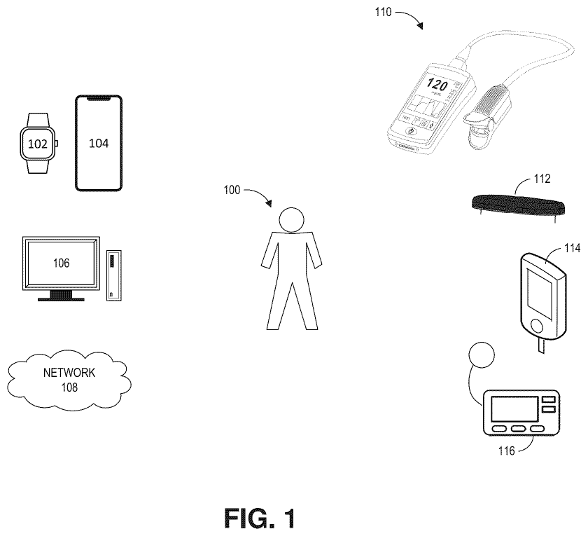

FIG. 1 illustrates an example environment in which an allergen detection system, such as described herein may operate. For example, as illustrated in FIG. 1, a person 100 may interact with a plurality of devices and/or sensors. The devices and/or sensors may be configured to detect, communicate, and/or display information associated with the person 100. The person 100 can be a patient. The person 100 can be a user. The person 100 can be an animal. Devices and/or sensors may include personal communication and/or smart devices, such as a smart watch 102, mobile phone 104, personal computing device 105, network or cloud based devices or processors 108. Devices and/or sensors may include medical monitoring devices, including but not limited to LED reflectance (or transmissive) sensor 110, such as a pulse oximeter, a minimally invasive sensor 112, a blood analyte monitor 114, acoustic monitoring sensor 116, camera, inflation cuff, impedance sensor, accelerometer, temperature sensor, and/or other physiological sensor.

The one or more devices and/or sensors may be configured to facilitate food logging or tracking. For example, one or more smart devices, such as a smartphone or smart watch may have an application for receiving food logging information. In some examples, the one or more devices and/or sensors may be configured to receive and log food related information from a person.

The one or more devices and/or sensors may be configured to measure aspects of a physiological state of a person 100. For example, the one or more devices and/or sensors may be configured to measure one or more physiological parameters, including but not limited to, respiration rate, throat diameter, bodily sounds, NiBP, pulse rate, hydration, galvanic skin response, pulse transit time, ECG, blood pressure, hemoglobin, oxygen content fractional oxygen saturation dis-hemoglobins (CO/MET), visual observation related behaviors and parameters, temperature, and/or the like. The one or more physiological parameters may be relevant to a physiological condition or state being tracked or monitored, such as an allergic reaction or other physiological state that necessitates medical attention in the immediate or non-immediate future.

One or more hardware processors may be configured to communicate with the one or more devices and/or sensors. In some examples, the one or more devices and/or sensors may be configured to process data from other devices and/or sensors to generate, for example, an alert or parameter associated with the monitored physiological state of the person 100. In some examples, one or more hardware processors may be associated with at least one wearable device 102, mobile device 104, cloud-based device 108, and/or personal computing device 106. One or more sensors and/or devices may be configured to communicate measured physiological parameters of the person 100 or signals associated with one or more physiological parameters of the person 100 to the one or more hardware processors. The one or more hardware processors may calculate one or more parameters based on at least one received physiological parameter and/or signal. The one or more hardware processers may analyze the one or more received and/or calculated physiological parameters to determine a physiological state of a person 100.

The one or more hardware processors may generate an alert, status, or action associated with the physiological state of the person 100 based on the determined physiological state of the person 100. The status, action, or alert may be used to communicate a medical emergency or need for medical attention in some cases. In some implementations, the one or more hardware processors may cause a medication delivery device to administer a medication responsive to a determined medical emergency. The medication delivery device may or may not be implemented into a monitoring device and/or part of a monitoring system. For example, a monitoring device may include a minimally invasive implant configured to monitor a person 100. The monitoring minimally invasive implant may include or be in communication with a medication delivery system, such as a medication pump and/or catheter.

Advantageously, utilization of a plurality of devices and/or sensors that may already be worn or associated with a person to identify a medical emergency can improve medical outcomes by catching a potential medical emergency, such as an allergic reaction, before a person 100 might otherwise seek out medical attention. For example, an allergic reaction can sometimes be caught based on subtle changes in a person's body that would otherwise not alert an outside human observer. Thus, medical monitoring and treatment is improved. Additionally, functionality of wearable devices and/or sensors configured to detect at least one physiological parameter of a person, such as a smart watch, may be improved to allow for further monitoring and identification of medical emergencies beyond mere health and fitness alerts and/or fall alerts.

Systems and methods described herein can include a system to track physiological measurements of a person that can be configured to compute a ranking and call to action for a person to learn about their food sensitivity and severity as well as be warned, offered alternate substitutions, or suggested ingredients to counter their reaction. In preparation for severe cases of reaction an individual could have a wearable pump with an automatic trigger to deliver a lifesaving injection of epinephrine.

Systems and methods described herein can include a food logging app that allows manual and/or automatic or semi-automatic food tracking (such as, for example, from a planned food tray, camera recording, QR code, or smart tray). The app can be paired with or configured to run on or in communication with one or more hardware processors. In some examples, the app may be paired with a smart device (such as but not limited to a smart watch, shirt or the like) having one or more smart sensors. These smart sensors can include but are not limited to light based reflective/transmissive, acoustic, radio, impedance or the like. The smart sensors may be configured to collect information associated with the patient. The information may include, but is not limited to physiological information or data, such as respiration rate, pulse rate, blood pressure, methemoglobin, EKG, heart rate variability, pulse rate variability, temperature, hydration, galvanic skin response, or others.

After each consumption of food, one or more hardware processors, such as referenced herein (sometimes referred to herein as a controller), may be configured to identify one or more biomarkers. The biomarker(s) may be associated with physiological response of a person to the consumed food. The biomarker(s) may be measured continuously (or sequentially) to track physiological response. During a calibration period (which may be during an initialization, by request, ongoing, or periodic), the controller may establish a baseline non-reactive response. The baseline response may include patterns, trends, or other markers in the physiological data referenced by the controller (such as the data obtained by the one or more sensors described herein). Once a normal non-reactive response and patterns are known at rest, they can be compared to delta changes after ingestion or exposure to food as quantified measurements that can then be weighted and ranked to allow a software interface (Smart phone, watch, VR/AR or the like) to present an actionable insight for the wearer or a caregiver, such as a guardian, clinician, or other person. The actionable insight can then be used to take an action related to the reaction. For example, the actionable insight may be that the wearer seems to have a sensitive reaction to cashew nuts present in this food bar or even trigger emergency response with a direct cellular connection.

### Example Allergen Identification

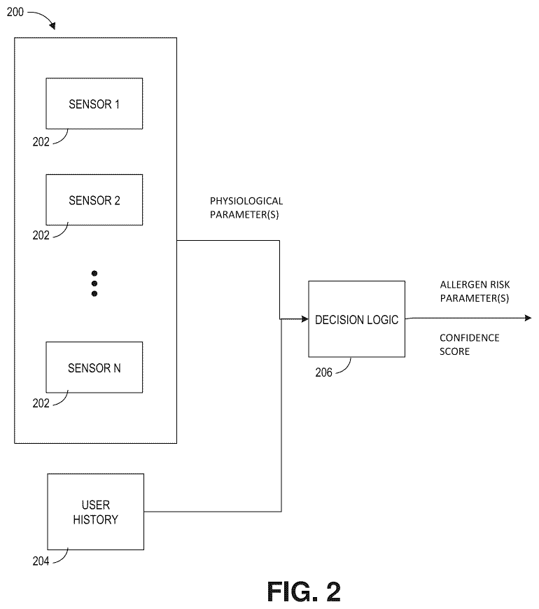

FIG. 2 illustrates an example process 200 for identifying an allergen based on physiological data. For example, as illustrated in FIG. 2, one or more sensors 202 can be configured to determine one or more physiological parameters of a patient. The one or more sensors may communicate with a wearable device.

The measured physiological parameters, as well as the user history 204 of the patient, can be input into the decision logic 206 of the systems and devices described herein. This decision logic 206 can output one or more potential allergens that caused the reaction of the patient. The risk level of these allergens can also be determined by the decision logic 206. The decision logic 206 may also output a confidence score associated with the one or more potential allergens. The decision logic 206 can comprise a series of steps to determine allergen risk parameters. The decision logic 206 can include neural networks or machine learning to determine allergen risk parameters.

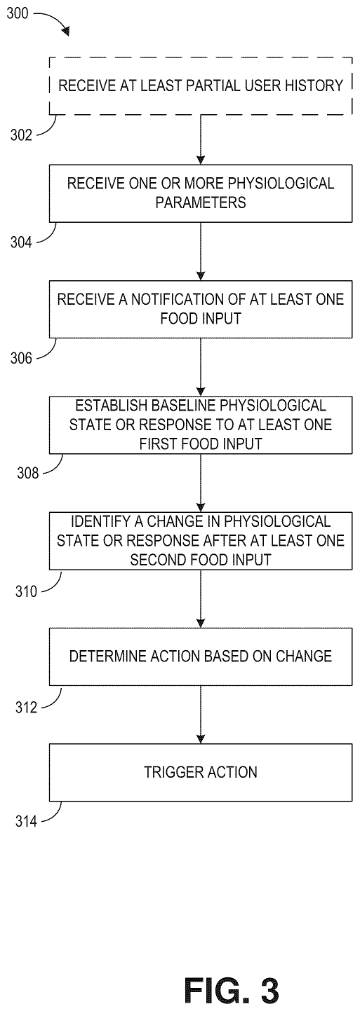

FIG. 3 illustrates an example process 300 of the decision logic processing signals from one or more sensors to determine an output. The process 300 can be conducted by a hardware processor. At block 302, the input can be configured to receive at least a partial user history of the patient. The at least partial user history can include previously known allergies of the patient. The at least partial user history can include previous reactions of the patient. The at least partial user history can include previously attained physiological parameters of the patient. The at least partial user history can include medical records of the patient. The at least partial user history can include a history of asthma. The at least partial user history can include a history of cardiovascular disease. The at least partial user history can include a history of chronic lung disease. The partial user history can be extracted from an electronic health record or an electronic medical record.

At block 304, the input can be configured to receive one or more physiological parameters. The physiological parameters can be determined by one or more sensors. The one or more sensors may include an LED reflectance sensor. The one or more sensors may include an LED transmissive sensor. The one or more sensors may include an acoustic sensor. The one or more sensors may include a camera. The sensors may include an intelligent inflation cuff. The sensors may include a pulse transit time from pulse oximeter. The one or more sensors may include an impedance sensor. The one or more sensors may include an accelerometer. The one or more sensors may include a pulse transit time sensor. The one or more sensors may include a thermometer. The one or more sensors may include a thermistor. The one or more sensors may comprise a minimally invasive disease management system. The sensors one or more may comprise one or more wearable devices.

With respect to block 304, the physiological parameters may include respiration rate (RR). The physiological parameters may include throat diameter. The physiological parameters may include sounds made during a digestion event, including chewing, swallowing, fragmentation in the stomach, small intestine absorption, large intestine exit, or other digestion events. The physiological parameters may include non-invasive blood pressure. The physiological parameters may include PR interval. The physiological parameters may include hydration. The physiological parameters may include galvanic skin response. The physiological parameters may include pulse transit time. The physiological parameters may include parameters measured by an electrocardiogram (ECG). The physiological parameters may include blood pressure. The physiological parameters may include hemoglobin level. The physiological parameters may include oxygen content. The physiological parameters may include oxygen content fractional oxygen saturation dis-hemoglobins (CO/MET). The physiological parameters may include metabolic rate. The physiological parameters may include physical appearance of hives or rash. The physiological parameters may include temperature of the skin. The physiological parameters may include internal temperature. The physiological parameters can be determined by measuring one or more biomarkers continuously or sequentially.

At block 306, the input can be configured to receive a notification of at least one food input. The food input can be determined by processing a signal of the one or more sensors. The food input can be manually input by the patient. The notification of the at least one food input can comprise an alert of the fact that food was consumed. The notification of the at least one food input can comprise information about the consumed food. The food input can be determined using a food logging application with manual, automatic, or semi-automatic tracking. The food input can be determined using a planned food tray. The food input can be determined using a camera recording of the food item. The camera can be controlled by the food logging application. The camera can be used to take a photograph or video that is uploaded to the food logging application. The food logging application can use augmented reality or virtual reality to track food consumed. The food input can be measured by wearable sensors or devices. The food input can be determined using a QR code input. The food input can be determined using a smart tray.

At block 308, the hardware processor can be configured to establish a baseline physiological state of the patient. The baseline physiological state can be a state in which the patient is not undergoing an allergic reaction. The hardware processor can be configured to establish a response to at least one first food input. The baseline physiological state of the patient can be determined using the at least partial user history. The baseline physiological state of the patient can be determined using the one or more physiological parameters. The baseline physiological state of the patient can be determined using a combination of the at least partial user history and the one or more physiological parameters. The baseline physiological state of the patient can be determined only using previous tracking of physiological parameters. For example, a person can be tracked for a set period of time or throughout a set number of food inputs to establish the baseline physiological state. Establishing the baseline physiological state can comprise calibrating the system. The systems and methods described herein can be configured to request that the patient cat and record certain foods before a baseline physiological state is set. For example, the systems and methods described herein can request that the patient cat 10 different food varieties to establish the baseline physiological state.

At block 310, the hardware processor can be configured to identify a change in the physiological state of the patient. The hardware processor can be configured to identify a physiological response of the patient or user to at least one second food input. The physiological response may include a change from a baseline value established by prior tracking and/or provided data, such as the at least partial user history. The change in physiological state or response to at least one second food input may be a change associated with an allergic reaction. For example, the change in physiological state or response can include constriction and tightening of the airways. The change in physiological state or response to at least one second food input may include a swollen throat. The change in physiological state or response to at least one second food input may include a sensation of a lump in the throat that makes it difficult to breathe. The change in physiological state or response to at least one second food input may include shock with a severe drop in blood pressure. The change in physiological state or response to at least one second food input may include rapid pulse. The change in physiological state or response to at least one second food input may include dizziness, lightheadedness, or loss of consciousness. The change in physiological state or response to at least one second food input may include urticaria or hives. The change in physiological state or response to at least one second food may include dilated pupils. The change in physiological state or response to at least one second food may include sweat. The change in physiological state or response to at least one second food may include dehydration. The change in physiological state or response to at least one second food may include change in breathing pace. The change in physiological state or response to at least one second food may include any of a cascading of mast cell activations and basophils crosslinking such as but not limited to immunoglobulin E, Calcium influx, or Sphingosine-1 phosphate.

At block 312, the hardware processor can be configured to determine an action based on the change in the physiological state. The action can include an alarm. The action can include an alarm that escalates if there is no response in 15 seconds. The action can include contacting someone in the patient's care circle. The action can include contacting emergency services. The action can include checking current hydration levels. The action can include triggering an auto-injection of medication. The auto-injection may be conducted by a wearable pump. The wearable pump can be configured to automatically inject the patient with a certain amount of medication. The auto-injection may be conducted by a combined wearable pump and continuous glucose monitoring system. The auto-injection may be conducted by a minimally invasive disease management system. The medication can include epinephrine. The medication can include an antihistamine. The medication can include diphenhydramine. The medication can include chlorpheniramine. The medication can include cetirizine. The medication can include desloratadine. The medication can include fexofenadine. The medication can include levocetirizine. The medication can include loratadine. The medication may comprise hydrocortisone. The action can include confirming the condition with the patient. The action can include having the patient speak a certain word or phrase to check their airway. The action can include alerting the patient about the allergic sensitivity. The action can include alerting the patient to the possibility of related allergic sensitivities. For example, if the patient has an allergic reaction to peanuts, the action can include alerting the patient to the possibility that the patient is allergic to cashews. The action can include instructing the patient to take medication. The action can include instructing the patient to drink water.

At block 314, the hardware processor can be configured to trigger the determined action. The hardware processor can trigger an alarm. The hardware processor can trigger an alarm that escalates if there is no response in 15 seconds. The hardware processor can contact someone in the patient's care circle. The hardware processor can contact emergency services. The hardware processor can check current hydration levels. The hardware processor can trigger an auto-injection of medication. The auto-injection may be conducted by a wearable pump. The wearable pump can be configured to automatically inject the patient with a certain amount of medication. The auto-injection may be conducted by a combined wearable pump and continuous glucose monitoring system. The auto-injection may be conducted by a minimally invasive disease management system. The medication can include epinephrine. The medication can include an antihistamine. The medication can include diphenhydramine. The medication can include chlorpheniramine. The medication can include cetirizine. The medication can include desloratadine. The medication can include fexofenadine. The medication can include levocetirizine. The medication can include loratadine. The medication may comprise hydrocortisone. The hardware processor can confirm the condition with the patient. The hardware processor can instruct the patient to speak a certain word or phrase to check their airway. The hardware processor can alert the patient about the allergic sensitivity. The hardware processor can alert the patient to the possibility of related allergic sensitivities. For example, if the patient has an allergic reaction to peanuts, the hardware processor can alert the patient to the possibility that the patient is allergic to cashews. The hardware processor can instruct the patient to take medication. The hardware processor can instruct the patient to drink water. The hardware processor can communicate with another device, for example a device of the patient, to communicate the change in physiological state and information measured by the one or more sensors. The hardware processor can record the data collected to an electronic health record or electronic medical record.

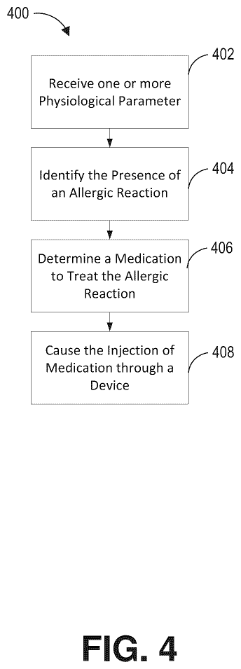

FIG. 4 illustrates an example process of using sensors to determine the presence of an allergic reaction and injecting a patient with medication. The process 400 can be conducted by a hardware processor. At block 402, the input can be configured to receive one or more physiological parameters. The physiological parameters can be determined by one or more sensors. The sensors may include an LED reflectance sensor. The sensors may include an LED transmissive sensor. The sensors may include an acoustic sensor. The sensors may include a camera. The sensors may include an intelligent inflation cuff. The sensors may include a pulse transit time from pulse oximeter. The sensors may include an impedance sensor. The sensors may include an accelerometer. The sensors may include a pulse transit time sensor. The sensors may include a thermometer. The sensors may include a thermistor. The sensors may comprise a minimally invasive disease management system. The sensors may comprise wearables.

The physiological parameters may include respiration rate (RR). The physiological parameters may include throat diameter. The physiological parameters may include sounds made during a digestion event, including chewing, swallowing, fragmentation in the stomach, small intestine absorption, large intestine exit, or other digestion events. The physiological parameters may include non-invasive blood pressure. The physiological parameters may include PR interval. The physiological parameters may include hydration. The physiological parameters may include galvanic skin response. The physiological parameters may include pulse transit time. The physiological parameters may include parameters measured by an electrocardiogram (ECG). The physiological parameters may include blood pressure. The physiological parameters may include hemoglobin level. The physiological parameters may include oxygen content. The physiological parameters may include oxygen content fractional oxygen saturation dis-hemoglobins (CO/MET). The physiological parameters may include metabolic rate. The physiological parameters may include physical appearance of hives or rash. The physiological parameters may include temperature of the skin. The physiological parameters may include internal temperature. The physiological parameters can be determined by measuring one or more biomarkers continuously or sequentially.

At block 404, the hardware processor can be configured to identify the presence of an allergic reaction. The presence of an allergic reaction can be identified based at least on the one or more physiological parameters received. Table 1, below, illustrates examples of anaphylaxis symptoms that may be measured by assessing certain parameters using certain sensors. The change in the parameters, or delta, can be used to identify allergic reactions, the time to cardiac arrest, and possible actions to help the patient. Increased risk factors can be considered by the systems and methods herein.

TABLE 1
illustrates an example of identifying anaphylaxis symptoms using changes in
physiological parameters measured by sensors, predicting risks, and determining an action.

| | | | | Time to | | |
|---|---|---|---|---|---|---|
| Anaphylaxis | | Sensor | | Cardiac | Increased | |
| Symptoms | Parameter | example | Delta | Arrest | risk | Action |
| Constriction and | RR and throat | LED reflectance | Lower | <30 Min | Previous | Alarm, |
| tightening of the | diameter, | (or transmissive) | SpO2, | | reaction, | Escalate if |
| airways | sound | sensor and | Increase | | Asthma or | no |
| | | acoustic | RR, | | chronic lung | response |
| | | monitoring, | Increase PR | | disease. | in 15 sec. |
| | | camera | | | Cardiovascular | Contact |
| A swollen throat or | RR and throat | LED reflectance | Increase | | | care circle, |
| the sensation of a | diameter, | sensor and | RR, lower | | | emergency |
| lump in your throat | sound | acoustic | SpO2 | | | services, |
| that makes it | | monitoring, | | | | Check |
| difficult to breathe | | camera | | | | current |
| Shock with a severe | NiBP, PR, | Intelligent | Decrease in | | | Hydration |
| drop in blood | Hydration, | inflation cuff, | BP | | | level, |
| pressure | Pulse Transit | pulse transit time | | | | Trigger |
| | time | from pulse ox. | | | | auto |
| | | | | | | injection |
| Rapid pulse | PR, ECG | LED reflectance | Increase of | | | of Epi |
| | | (or transmissive) | PR of X | | | dose, |
| | | sensor, | bpm in X | | | confirm |
| | | impendence, | time | | | with |
| | | camera | | | | patient |
| Dizziness, | Blood | Accelerometer, | Drop in BP | | | condition |
| lightheadedness or | Pressure, | Pulse Transit | of X, | | | having |
| loss of | hydration or | Time | Hyrdration | | | them |
| consciousness | hemoglobin, | | status | | | speak their |
| | oxygen | | change of x, | | | name to |
| | content | | drop in Hb, | | | check |
| | fractional | | drop in | | | airway |
| | oxygen | | FSpO2, | | | clear. |
| | saturation dis- | | increase in | | | |
| | hemoglobins | | CO/MET | | | |
| | (CO/MET) | | | | | |
| Urticaria (hives) | Visual | Thermistor, | Increase | | | |
| | observation, | camera | skin | | | |
| | hydration, | | temperature | | | |
| | temperature | | | | | |

The hardware processor can establish a baseline physiological state or a response to a first food. The allergic reaction can be identified based at least on a change in physiological state or response to at least one second food. The change in physiological state or response to at least one second food input may include constriction and tightening of the airways. The change in physiological state or response to at least one second food input may include a swollen throat. The change in physiological state or response to at least one second food input may include a sensation of a lump in the throat that makes it difficult to breathe. The change in physiological state or response to at least one second food input may include shock with a severe drop in blood pressure. The change in physiological state or response to at least one second food input may include rapid pulse. The change in physiological state or response to at least one second food input may include dizziness, lightheadedness, or loss of consciousness. The change in physiological state or response to at least one second food input may include urticaria or hives. The change in physiological state or response to at least one second food may include dilated pupils. The change in physiological state or response to at least one second food may include sweat. The change in physiological state or response to at least one second food may include dehydration. The change in physiological state or response to at least one second food may include change in breathing pace. The change in physiological state or response to at least one second food may include any of a cascading of mast cell activations and basophils crosslinking such as but not limited to immunoglobulin E, Calcium influx, or Sphingosine-1 phosphate.

At block 406, the hardware processor can be configured to determine a medication to treat the allergic reaction. The medication can include epinephrine. The medication can include an antihistamine. The medication can include diphenhydramine. The medication can include chlorpheniramine. The medication can include cetirizine. The medication can include desloratadine. The medication can include fexofenadine. The medication can include levocetirizine. The medication can include loratadine. The medication may comprise hydrocortisone. The determination of a medication can include determining an amount of a medication that would be beneficial. The medication type may be predetermined. The amount of medication may be predetermined.

At block 408, the hardware processor can be configured to cause an injection of a medication through a device. The injection may be an auto-injection. The auto-injection may be conducted by a wearable pump. The wearable pump can be configured to automatically inject the patient with a certain amount of medication. The auto-injection may be conducted by a combined wearable pump and continuous glucose monitoring system. The auto-injection may be conducted by a minimally invasive disease management system. The injection may comprise epinephrine. The injection may comprise an antihistamine. The injection may comprise hydrocortisone.

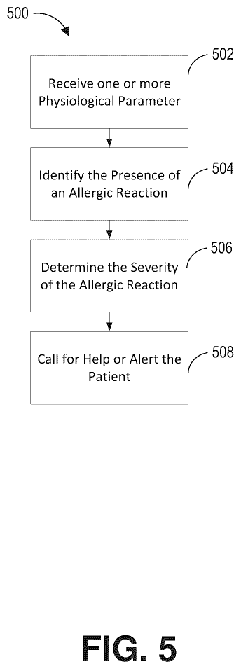

FIG. 5 illustrates an example process of using sensors to determine the presence of an allergic reaction and alerting a patient or calling for help. The process 500 can be conducted by a hardware processor. At block 502, the input can be configured to receive one or more physiological parameters. The physiological parameters can be determined by one or more sensors. The sensors may include an LED reflectance sensor. The sensors may include an LED transmissive sensor. The sensors may include an acoustic sensor. The sensors may include a camera. The sensors may include an intelligent inflation cuff. The sensors may include a pulse transit time from pulse oximeter. The sensors may include an impedance sensor. The sensors may include an accelerometer. The sensors may include a pulse transit time sensor. The sensors may include a thermometer. The sensors may include a thermistor. The sensors may comprise a minimally invasive disease management system. The sensors may comprise wearables.

The physiological parameters may include respiration rate (RR). The physiological parameters may include throat diameter. The physiological parameters may include sounds made during a digestion event, including chewing, swallowing, fragmentation in the stomach, small intestine absorption, large intestine exit, or other digestion events. The physiological parameters may include non-invasive blood pressure. The physiological parameters may include PR interval. The physiological parameters may include hydration. The physiological parameters may include pulse transit time. The physiological parameters may include parameters measured by an electrocardiogram (ECG). The physiological parameters may include blood pressure. The physiological parameters may include hemoglobin level. The physiological parameters may include oxygen content. The physiological parameters may include oxygen content fractional oxygen saturation dis-hemoglobins (CO/MET). The physiological parameters may include metabolic rate. The physiological parameters may include physical appearance of hives or rash. The physiological parameters may include temperature of the skin. The physiological parameters may include internal temperature.

At block 504, the hardware processor can be configured to identify the presence of an allergic reaction. The presence of an allergic reaction can be identified based at least on the one or more physiological parameters received. The hardware processor can establish a baseline physiological state or a response to a first food. The allergic reaction can be identified based at least on a change in physiological state or response to at least one second food. The change in physiological state or response to at least one second food input may include constriction and tightening of the airways. The change in physiological state or response to at least one second food input may include a swollen throat. The change in physiological state or response to at least one second food input may include a sensation of a lump in the throat that makes it difficult to breathe. The change in physiological state or response to at least one second food input may include shock with a severe drop in blood pressure. The change in physiological state or response to at least one second food input may include rapid pulse. The change in physiological state or response to at least one second food input may include dizziness, lightheadedness, or loss of consciousness. The change in physiological state or response to at least one second food input may include urticaria or hives. The change in physiological state or response to at least one second food may include dilated pupils. The change in physiological state or response to at least one second food may include sweat. The change in physiological state or response to at least one second food may include dehydration. The change in physiological state or response to at least one second food may include change in breathing pace. The change in physiological state or response to at least one second food may include any of a cascading of mast cell activations and basophils crosslinking such as but not limited to immunoglobulin E, Calcium influx, or Sphingosine-1 phosphate.

At block 506, the hardware processor can be configured to determine the severity of the allergic reaction. The severity of the allergic reaction can be determined based at least in part on the one or more physiological parameters received. The severity of the allergic reaction can be determined based at least in part on the type of food consumed. The severity of the allergic reaction can be determined based at least in part on the extremity of the measurements by the sensor. The severity of the allergic reaction can be determined based at least in part on past allergic reactions of the patient. The severity of the allergic reaction can be determined based at least in part on medical of the patient. The severity of the allergic reaction can be determined by the amount of time that has passed since the food item was consumed. The severity of the allergic reaction may be considered lower when more time has passed if the physiological parameters do not indicate a serious reaction. The severity of the allergic reaction may be considered lower when thirty minutes has passed if the physiological parameters do not indicate a serious reaction. The severity of the allergic reaction may be considered lower when one hour has passed if the physiological parameters do not indicate a serious reaction.

At block 508, the hardware processor can be configured to call for help or alert the patient to the presence of an allergic reaction. The systems and methods herein can be configured to alert the patient as to the presence of the reaction when the reaction is not severe. Alternatively, the systems and methods herein can be configured to call for help when the reaction is not severe. The systems and methods herein can be configured to call for help when the reaction is severe. Alternatively, the systems and methods herein can be configured to alert the patient as to the presence of the reaction when the reaction is severe. Calling for help can comprise calling someone in the patient's care circle. Calling for help can comprise calling emergency services. Calling for help can comprise calling physician.

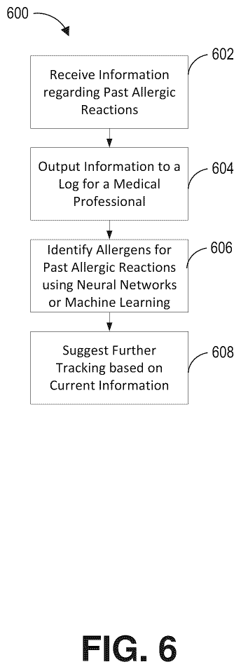

FIG. 6 illustrates an example process of using neural networks or machine learning to identify allergic reactions. The process 600 can be conducted by a hardware processor. At block 602, the hardware processor can be configured to receive information regarding past allergic reactions. The information regarding past allergic reactions can be attributed the patient. The information regarding past allergic reactions can be attributed to a group of other people. The information regarding allergic reactions can comprise the presence of allergic reactions in combination with other attributes. The information regarding allergic reactions can comprise the severity of allergic reactions in combination with other attributes.

At block 604, the hardware processor can be configured to output information to a log for a medical professional. The information output can comprise information regarding allergic reactions of the patient. The information regarding allergic reactions can comprise the presence of allergic reactions in combination with other attributes. The information regarding allergic reactions can comprise the severity of allergic reactions in combination with other attributes. The log may comprise mild reactions. A log comprising mild reactions may be output to a medical professional to determine treatment options or predict increased or decreased severity of the allergic sensitivity in the future.

At block 606, the hardware processor can be configured to identify allergens for past allergic reactions using neural networks or machine learning. The hardware processor can be configured to use artificial intelligence. The neural networks or machine learning can be trained using a training set of other people's physiological parameters and allergic sensitivities. The neural networks or machine learning may output allergic sensitivities of a user. The neural networks or machine learning may output confidence scores related to potential allergic sensitivities of a user. Allergic reactions can be identified using machine learning techniques with the inputs being the parameters being tracked or past allergic reactions to similar foods. For example, if the patient had an allergic reaction to cashews, the machine learning techniques may predict whether the patient has an allergic sensitivity to peanuts.

At block 608, the hardware processor can be configured to suggest further tracking based on current information. The hardware processor can use information obtained to adjust criteria for determining an allergic reaction. For example, if the neural networks or machine learning output indicates that the patient may have an allergic sensitivity to cashews, the hardware processor may lower its threshold for determining that the patient is undergoing an allergic reaction when the patient consumes cashews or related food items.

### Additional Examples

Disclosed herein are additional examples of systems, methods, and/or devices described herein. The disclosed examples are non-limiting. Any of the disclosed examples may be combined in whole or in part. Any of the disclosed examples may include different, fewer, or more aspects than explicitly described.
Example 1. A system for determining an allergic sensitivity of a patient, the system comprising: at least one sensor configured to measure one or more physiological parameters of a patient; an input configured to receive: at least one signal from the at least one sensor; and at least partial user history of the patient; and one or more hardware processors configured to: process the at least one signal from the at least one sensor and the at least partial user history of the patient; establish a baseline physiological state of the patient or a response to at least one first potential allergen; identify a change in a physiological state of the patient or a response to at least one second potential allergen; determine an allergic reaction of the patient based on the change in the physiological state of the patient or a difference between the response to the at least one first potential allergen and the response to the at least one second potential allergen; determine a response action to the allergic reaction of the patient; and implement the response action.
Example 2. The system of Example 1, wherein the at least one first potential allergen is at least one food item.
Example 3. The system of Example 1, wherein the at least one second potential allergen is at least one food item.
Example 4. The system of Example 1, wherein the at least one first potential allergen is at least one medication.
Example 5. The system of Example 1, wherein the at least one second potential allergen is at least one medication.
Example 6. The system of Example 1, wherein the at least one first potential allergen is at least one animal fur, skin, saliva or urine.
Example 7. The system of Example 1, wherein the at least one second potential allergen is at least one animal fur, skin, saliva or urine.
Example 8. The system of Example 1, wherein the at least one first potential allergen is at least one pollen.
Example 9. The system of Example 1, wherein the at least one second potential allergen is at least one pollen.
Example 10. The system of Example 1, wherein the at least one first potential allergen is at least one dust.
Example 11. The system of Example 1, wherein the at least one second potential allergen is at least one dust.
Example 12. The system of Example 1, wherein the response action comprises causing a medication injection device to inject the patient with medication.
Example 13. The system of Example 12, wherein the medication injection device is a wearable pump configured to inject the patient with epinephrine.
Example 14. The system of Example 12, wherein the medication injection device is a wearable pump configured to inject the patient with an antihistamine.
Example 15. The system of Example 12, wherein the medication injection device is a wearable pump configured to inject the patient with hydrocortisone.
Example 16. The system of Example 1, wherein the response action comprises instructing the patient to take a medication.
Example 17. The system of Example 16, wherein the medication comprises epinephrine.
Example 18. The system of Example 16, wherein the medication comprises an antihistamine.
Example 19. The system of Example 16, wherein the medication comprises hydrocortisone.
Example 20. The system of Example 1, wherein the response action comprises instructing the patient to drink water.
Example 21. The system of Example 1, wherein the at least one sensor comprises at least one wearable sensor.
Example 22. The system of Example 21, wherein the at least one wearable sensor comprises a smartwatch.
Example 23. The system of Example 1, wherein the at least partial user history of the patient comprises medical records.
Example 24. The system of Example 1, wherein the at least partial user history of the patient comprises one or more prior allergic reactions.
Example 25. The system of Example 1, wherein the at least partial user history of the patient comprises previously known allergens of the patient.
Example 26. The system of Example 1, wherein the at least partial user history of the patient comprises a history of asthma, cardiovascular disease, or chronic lung disease.
Example 27. The system of Example 1, wherein the baseline physiological state of the patient comprises a nonreactive state.
Example 28. The system of Example 1, wherein the baseline physiological state of the patient comprises a state in which the patient has not contacted potential allergens.
Example 29. The system of Example 1, wherein the one or more hardware processors is configured to determine the patient has an allergic reaction when the difference between the response to the at least one first potential allergen and the response to the at least one second potential allergen is greater than an allergy threshold.
Example 30. The system of Example 1, wherein the one or more hardware processors is configured to determine the patient has an allergic reaction when the change in the physiological state of the patient is greater than an allergy threshold.
Example 31. The system of Example 1, wherein the one or more hardware processors is configured to characterize a severity of the allergic reaction based on whether the difference between the response to the at least one first potential allergen and the response to the at least one second potential allergen is greater than one or more of a plurality of severity thresholds.
Example 32. The system of Example 1, wherein the one or more hardware processors is configured to characterize a severity of the allergic reaction based on whether the change in the physiological state of the patient is greater than one or more of a plurality of severity thresholds.
Example 33. The system of Example 1, wherein the response action comprises alerting the patient of the allergic reaction.
Example 34. The system of Example 33, wherein alerting the patient of the allergic reaction comprises notifying the patient on a display of a user device.
Example 35. The system of Example 33, wherein alerting the patient of the allergic reaction comprises notifying the patient on a display of a smartwatch.
Example 36. The system of Example 1, wherein the response action comprises alerting a contact of the patient of the allergic reaction.
Example 37. The system of Example 1, wherein the response action comprises alerting an emergency service of the allergic reaction.
Example 38. The system of Example 1, wherein the at least one sensor comprises imaging sensors, photoplethysmography sensors, acoustic sensors, electromyogram sensors, electroencephalogram sensors, or motion sensors.
Example 39. The system of Example 1, wherein the one or more physiological parameters of the patient comprises respiration rate.
Example 40. The system of Example 1, wherein the one or more physiological parameters of the patient comprises throat diameter.
Example 41. The system of Example 1, wherein the one or more physiological parameters of the patient comprises bodily sounds.
Example 42. The system of Example 1, wherein the one or more physiological parameters of the patient comprises non-invasive blood pressure.
Example 43. The system of Example 1, wherein the one or more physiological parameters of the patient comprises pulse rate.
Example 44. The system of Example 1, wherein the one or more physiological parameters of the patient comprises hydration.
Example 45. The system of Example 1, wherein the one or more physiological parameters of the patient comprises galvanic skin response.
Example 46. The system of Example 1, wherein the one or more physiological parameters of the patient comprises pulse transit time.
Example 47. The system of Example 1, wherein the one or more physiological parameters of the patient comprises parameters measured by an electrocardiogram.
Example 48. The system of Example 1, wherein the one or more physiological parameters of the patient comprises blood pressure.
Example 49. The system of Example 1, wherein the one or more physiological parameters of the patient comprises hemoglobin.
Example 50. The system of Example 1, wherein the one or more physiological parameters of the patient comprises oxygen content fractional oxygen saturation dis-hemoglobins.
Example 51. The system of Example 1, wherein the one or more physiological parameters of the patient comprises metabolic rate.
Example 52. The system of Example 1, wherein the one or more physiological parameters of the patient comprises visual observation related behaviors and parameters.
Example 53. The system of Example 1, wherein the one or more physiological parameters of the patient comprises temperature of skin.
Example 54. The system of Example 1, wherein the one or more physiological parameters of the patient comprises internal temperature.
Example 55. The system of Example 1, wherein the one or more physiological parameters of the patient comprises appearance of hives or rash.
Example 56. The system of Example 1, wherein the one or more physiological parameters of the patient comprises parameters related to symptoms of allergic reactions.
Example 57. A method of determining an allergic sensitivity of a patient, the method comprising: receiving at least one signal from at least one sensor configured to measure one or more physiological parameters of the patient; receiving at least partial user history of the patient; processing the at least one signal from the at least one sensor and the at least partial user history of the patient; establishing a baseline physiological state of the patient or a response to at least one first potential allergen; identifying a change in a physiological state of the patient or a response to at least one second potential allergen; determining an allergic reaction of the patient based on the change in the physiological state of the patient or a difference between the response to the at least one first potential allergen and the response to the at least one second potential allergen; determining a response action to the allergic reaction of the patient; and implementing the response action.
Example 58. The method of Example 57, wherein the at least one first potential allergen is at least one food item.
Example 59. The method of Example 57, wherein the at least one second potential allergen is at least one food item.
Example 60. The method of Example 57, wherein the at least one first potential allergen is at least one medication.
Example 61. The method of Example 57, wherein the at least one second potential allergen is at least one medication.
Example 62. The method of Example 57, wherein the at least one first potential allergen is at least one animal fur, skin, saliva or urine.
Example 63. The method of Example 57, wherein the at least one second potential allergen is at least one animal fur, skin, saliva or urine.
Example 64. The method of Example 57, wherein the at least one first potential allergen is at least one pollen.
Example 65. The method of Example 57, wherein the at least one second potential allergen is at least one pollen.
Example 66. The method of Example 57, wherein the at least one first potential allergen is at least one dust.
Example 67. The method of Example 57, wherein the at least one second potential allergen is at least one dust.
Example 68. The method of Example 57, wherein the response action comprises causing a medication injection device to inject the patient with medication.
Example 69. The method of Example 68, wherein the medication injection device is a wearable pump configured to inject the patient with epinephrine.
Example 70. The method of Example 68, wherein the medication injection device is a wearable pump configured to inject the patient with an antihistamine.
Example 71. The method of Example 68, wherein the medication injection device is a wearable pump configured to inject the patient with hydrocortisone.
Example 72. The method of Example 57, wherein the response action comprises instructing the patient to take a medication.
Example 73. The method of Example 72, wherein the medication comprises epinephrine.
Example 74. The method of Example 72, wherein the medication comprises an antihistamine.
Example 75. The method of Example 72, wherein the medication comprises hydrocortisone.
Example 76. The method of Example 57, wherein the response action comprises instructing the patient to drink water.
Example 77. The method of Example 57, wherein the at least one sensor comprises at least one wearable sensor.
Example 78. The method of Example 77, wherein the at least one wearable sensor comprises a smartwatch.
Example 79. The method of Example 57, wherein the at least partial user history of the patient comprises medical records.
Example 80. The method of Example 57, wherein the at least partial user history of the patient comprises one or more prior allergic reactions.
Example 81. The method of Example 57, wherein the at least partial user history of the patient comprises previously known allergens of the patient.
Example 82. The method of Example 57, wherein the at least partial user history of the patient comprises a history of asthma, cardiovascular disease, or chronic lung disease.
Example 83. The method of Example 57, wherein the baseline physiological state of the patient comprises a nonreactive state.
Example 84. The method of Example 57, wherein the baseline physiological state of the patient comprises a state in which the patient has not contacted potential allergens.
Example 85. The method of Example 57, further configured to determine the patient has an allergic reaction when the difference between the response to the at least one first potential allergen and the response to the at least one second potential allergen is greater than an allergy threshold.
Example 86. The method of Example 57, further configured to determine the patient has an allergic reaction when the change in the physiological state of the patient is greater than an allergy threshold.
Example 87. The method of Example 57, further configured to characterize a severity of the allergic reaction based on whether the difference between the response to the at least one first potential allergen and the response to the at least one second potential allergen is greater than one or more of a plurality of severity thresholds.
Example 88. The method of Example 57, further configured to characterize a severity of the allergic reaction based on whether the change in the physiological state of the patient is greater than one or more of a plurality of severity thresholds.
Example 89. The method of Example 57, wherein the response action comprises alerting the patient of the allergic reaction.
Example 90. The method of Example 89, wherein alerting the patient of the allergic reaction comprises notifying the patient on a display of a user device.
Example 91. The method of Example 89, wherein alerting the patient of the allergic reaction comprises notifying the patient on a display of a smartwatch.
Example 92. The method of Example 57, wherein the response action comprises alerting a contact of the patient of the allergic reaction.
Example 93. The method of Example 57, wherein the response action comprises alerting an emergency service of the allergic reaction.
Example 94. The method of Example 57, wherein the at least one sensor comprises imaging sensors, photoplethysmography sensors, acoustic sensors, electromyogram sensors, electroencephalogram sensors, or motion sensors.
Example 95. The method of Example 57, wherein the one or more physiological parameters of the patient comprises respiration rate.
Example 96. The method of Example 57, wherein the one or more physiological parameters of the patient comprises throat diameter.
Example 97. The method of Example 57, wherein the one or more physiological parameters of the patient comprises bodily sounds.
Example 98. The method of Example 57, wherein the one or more physiological parameters of the patient comprises non-invasive blood pressure.
Example 99. The method of Example 57, wherein the one or more physiological parameters of the patient comprises pulse rate.
Example 100. The method of Example 57, wherein the one or more physiological parameters of the patient comprises hydration.
Example 101. The method of Example 57, wherein the one or more physiological parameters of the patient comprises galvanic skin response.
Example 102. The method of Example 57, wherein the one or more physiological parameters of the patient comprises pulse transit time.
Example 103. The method of Example 57, wherein the one or more physiological parameters of the patient comprises parameters measured by an electrocardiogram.
Example 104. The method of Example 57, wherein the one or more physiological parameters of the patient comprises blood pressure.
Example 105. The method of Example 57, wherein the one or more physiological parameters of the patient comprises hemoglobin.
Example 106. The method of Example 57, wherein the one or more physiological parameters of the patient comprises oxygen content fractional oxygen saturation dis-hemoglobins.
Example 107. The method of Example 57, wherein the one or more physiological parameters of the patient comprises metabolic rate.
Example 108. The method of Example 57, wherein the one or more physiological parameters of the patient comprises visual observation related behaviors and parameters.
Example 109. The method of Example 57, wherein the one or more physiological parameters of the patient comprises temperature of skin.
Example 110. The method of Example 57, wherein the one or more physiological parameters of the patient comprises internal temperature.
Example 111. The method of Example 57, wherein the one or more physiological parameters of the patient comprises appearance of hives or rash.
Example 112. The method of Example 57, wherein the one or more physiological parameters of the patient comprises parameters related to symptoms of allergic reactions.
Example 113. A method for determining an allergic reaction of a patient, the method comprising: receiving at least one first signal from at least one sensor configured to measure physiological parameters of the patient; receiving at least partial user history of the patient; displaying a graphical user interface (GUI) on a screen of a user device coupled to at least one sensor, the GUI containing at least one physiological parameter at a baseline physiological state of the patient or in response to at least one first potential allergen; receiving at least one second signal from at least one sensor configured to measure physiological parameters of the patient; displaying a GUI on a screen of a user device coupled to at least one sensor, the GUI containing: an indication of presence of an allergic reaction of the patient; at least one physiological parameter at a baseline physiological state of the patient or in response to at least one first potential allergen; at least one physiological parameter at a changed physiological state of the patient or in response to at least one second potential allergen; and an indication of severity of the allergic reaction of the patient; wherein the user device comprises a computer processor and an electronic storage medium.
Example 114. A medication injection device, the medication injection device comprising: a wearable pump housing configured to receive a medication container; the medication container configured to be inserted into the wearable pump housing, the medication container having a dispensing end through which medication is dispensed, wherein the dispensing end of the medication container is configured to align with a frontal end of the wearable pump housing when the medication container is inserted into the wearable pump housing; a piston rod at a distal end of the wearable pump housing; a battery inside the wearable pump housing, the battery configured to supply electrical power to a piston rod pushing system; a hardware processor in communication with the piston rod pushing system, the hardware processor configured to determine when a patient requires medication for an allergic reaction; and the piston rod pushing system connected to the piston rod, the piston rod pushing system configured to communicate with the hardware processor to receive instructions, the piston rod pushing system further configured to push the piston rod toward the frontal end of the wearable pump housing when the hardware processor determines the patient requires medication for the allergic reaction, causing the medication to be dispensed.
Example 115. The device of Example 114, wherein the medication comprises epinephrine.
Example 116. The device of Example 114, wherein the medication comprises an antihistamine.
Example 117. The device of Example 114, wherein the medication comprises hydrocortisone.
Example 118. The device of Example 114, wherein the hardware processor determines the allergic reaction based on input from at least one sensor.

### Terminology

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps, but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Any methods disclosed herein need not be performed in the order recited. The methods disclosed herein include certain actions taken by a practitioner; however, they can also include any third-party instruction of those actions, either expressly or by implication.

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A system for determining an allergic sensitivity of a patient, the system comprising:
at least one sensor configured to measure one or more physiological parameters of a patient;
an input configured to receive:
at least one signal from the at least one sensor; and
at least partial user history of the patient;
one or more hardware processors configured to:
process the at least one signal from the at least one sensor and the at least partial user history of the patient;
establish a baseline physiological state of the patient or a response to at least one first potential allergen;
identify a change in a physiological state of the patient or a response to at least one second potential allergen;
determine an allergic reaction of the patient based on the change in the physiological state of the patient or a difference between the response to the at least one first potential allergen and the response to the at least one second potential allergen; and
a medication injection device configured to inject the patient with medication in response to the allergic reaction.

2. The system of claim 1, wherein the medication injection device is a wearable pump configured to inject the patient with epinephrine.

3. The system of claim 1, wherein the at least partial user history is extracted from an electronic medical record or an electronic health record.

4. The system of claim 1, wherein the one or more hardware processors is further configured to instruct the patient to take a medication in response to the allergic reaction.

5. The system of claim 1, wherein the at least one sensor comprises at least one wearable sensor.

6. The system of claim 5, wherein the at least one wearable sensor comprises a smartwatch.

7. The system of claim 1, wherein the one or more hardware processors is configured to determine the patient has the allergic reaction when the difference between the response to the at least one first potential allergen and the response to the at least one second potential allergen is greater than an allergy threshold.

8. The system of claim 1, wherein the one or more hardware processors is configured to characterize a severity of the allergic reaction based on whether the difference between the response to the at least one first potential allergen and the response to the at least one second potential allergen is greater than one or more of a plurality of severity thresholds.

9. The system of claim 1, further comprising recording information about the allergic reaction to an electronic medical record or an electronic health record.

10. The system of claim 1, wherein the at least one sensor comprises imaging sensors, photoplethysmography sensors, acoustic sensors, electromyogram sensors, electroencephalogram sensors, or motion sensors.

11. A method of determining an allergic sensitivity of a patient, the method comprising:
receiving at least one signal from at least one sensor configured to measure one or more physiological parameters of the patient;
receiving at least partial user history of the patient;
processing the at least one signal from the at least one sensor and the at least partial user history of the patient;
establishing a baseline physiological state of the patient or a response to at least one first potential allergen;
identifying a change in a physiological state of the patient or a response to at least one second potential allergen;
determining an allergic reaction of the patient based on the change in the physiological state of the patient or a difference between the response to the at least one first potential allergen and the response to the at least one second potential allergen; and
injecting, with a medication injection device, the patient with medication in response to the allergic reaction.

12. The method of claim 11, further comprising extracting the at least partial user history of the patient from an electronic medical record or an electronic health record.

13. The method of claim 11, wherein the medication is epinephrine.

14. The method of claim 11, further comprising instructing the patient to take a medication.

15. The method of claim 11, wherein determining the allergic reaction of the patient comprises determining that the difference between the response to the at least one first potential allergen and the response to the at least one second potential allergen is greater than an allergy threshold.

16. The method of claim 11, further comprising alerting the patient of the allergic reaction.

17. The method of claim 16, wherein alerting the patient of the allergic reaction comprises communicating information about the allergic reaction to a user device.

18. The method of claim 11, further comprising recording information about the allergic reaction to an electronic medical record or an electronic health record.

19. The method of claim 11, further comprising alerting an emergency service of the allergic reaction.

20. The method of claim 11, wherein receiving at least one signal from the at least one sensor comprises receiving at least one signal from imaging sensors, photoplethysmography sensors, acoustic sensors, electromyogram sensors, electroencephalogram sensors, or motion sensors.
